(19) United States
(12) Patent Application Publication
(10) Pub. No.: US 2007/0038269 A1
(43) Pub. Date: Feb. 15, 2007

(54) Therapeutic method and apparatus

(75) Inventor: Colin Whitehurst, Cheshire (GB)

Correspondence Address: STERNE, KESSLER, GOLDSTEIN & FOX PLLC, 1100 NEW YORK AVENUE, N.W., WASHINGTON, DC, 20005, US

(73) Assignee: Photo Therapeutics Ltd.

(21) Appl. No.: 10/547,081

(22) PCT Filed: Feb. 26, 2003
(86) PCT No.: PCT/GB03/00834

§ 371 Date: Jul. 26, 2006

(51) Int. Cl. A61N 5/06

(52) U.S. Cl. 607/88

(57) ABSTRACT

A method of promoting healing of wounds or tissue damage comprises irradiating the affected area with polychromatic radiation substantially within the wavelength range of 405 to 904 nm. Preferably, the radiation has substantial intensity at a wavelength of one or more of 633, 680 or 780 nm, so as to stimulate DNA/RNA synthesis. Preferably, the radiation has substantial intensity at a wavelength of 750 nm so A as to promote protein synthesis. Preferably, the radiation has substantial intensity at 890 nm, so as to cause increased cell proliferation. Preferably, the radiation has substantial intensity at a wavelength of 880 nm, so as to inhibit fibroblast proliferation. Alternatively, the radiation has substantial intensity at 820 nm and/or 870 nm, so as to enhance the release of stimulating factors. A method of treating hypopigmentary skin disorders, such as vitiligo, comprises irradiating a hypopigmented area of a patient's skin with low intensity ultraviolet radiation. Preferably, the low intensity ultraviolet radiation has substantial intensity at a wavelength of at least one of 365, 404 and 434 mn. Preferably, the radiation has a substantial intensity at a wavelength of approximately 760 nm. The radiation may be provided by an array of discrete light-emitting diodes arranged to emit the polychromatic light or low intensity ultraviolet light.

### FIELD OF THE INVENTION

The present invention relates to therapeutic methods and apparatus for wound healing, tissue repair and/or cosmetic dermatological treatment, or the treatment of hypopigmentation.

### BACKGROUND OF THE INVENTION

Wound healing is a complex biochemical process that commences immediately after tissue injury. A coagulation phase is characterized by low oxygen tension and the formation of platelet plugs. Macrophages, polymorphnuclear neutrophils and lymphocytes appear during the inflannnatory phase for debris and infection control, and growth factors are secreted for fibroplasias, angiogenesis and re-epithelialisation.

It would be desirable to achieve a method of treatment, and a device for use in such a method, for enhancing wound repair and/or tissue repair, or for cosmetic dermatological treatment.

The documents WO 95/19809 and WO95/19810 disclose devices for healing wounds and sores by means of a light-emitting element which emits pulsed light infrared during a first period and pulsed red light during a second period. According to this document, it is extremely important that the treatment is carried out in the order infrared light followed by visible light. The light-emitting element includes discrete infrared and red light emitting diodes. In an example, the red emission is at 660 nm and the infrared at 950 nm.

Certain hypopigmentary skin disorders, such as vitiligo, are characterised by a loss of melanocytes from the epidermis, which results in the absence of melanin, i.e. depigmentation. The problem of vitiligo would be essentially solved if there were a medication that is well tolerated in children, adults and pregnant women, and that would halt the progression of the depigmentation.

### STATEMENT OF THE INVENTION

According to a first aspect of the present invention, there is provided a method of promoting healing of wounds or tissue damage by irradiating the affected area with polychromatic radiation substantially within the wavelength range of 405 to 904 mn. Preferably, the radiation has substantial intensity at a wavelength of one or more of 633, 680 or 780 nm, so as to stimulate DNA/RNA synthesis. Preferably, the radiation has substantial intensity at a wavelength of 750 nm so as to promote protein synthesis/Preferably, the radiation has substantial intensity at 890 nm, so as to cause increased cell proliferation. Preferably, the radiation has substantial intensity at a wavelength of 880 nm, so as to inhibit fibroblast proliferation. Alternatively, the radiation has substantial intensity at 820 nm and/or 870 nm, so as to enhance the release of stimulating factors.

According to a second aspect of the present invention, there is provided a method of treating hypopigmentary skin disorders, such as vitiligo, by irradiating a hypopigmented area of a patient's skin with low intensity ultraviolet radiation. Preferably, the low intensity ultraviolet radiation has substantial intensity at a wavelength of at least one of 365, 404 and 434 nm. Preferably, the radiation has a substantial intensity at a wavelength of approximately 760 nm.

According to a third aspect of the present invention, there is provided a light source for carrying out the method of the first aspect, including an array of light-emitting diodes arranged to emit the polychromatic light.

According to a fourth aspect of the present invention, there is provided a light source for carrying out the method of the second aspect, the light source including an array of light-emitting diodes arranged to emit the low intensity ultraviolet radiation.

In either the third or fourth aspects, the configuration of the plurality of arrays is preferably adjustable by the user. The plurality of arrays may be mounted on an adjustable arm.

### BRIEF DESCRIPTION OF THE DRAWINGS

Specific embodiments of the present invention will now be described with reference to the accompanying drawings, in which:

FIG. 1 is schematic side view of a therapeutic light source in embodiments of the invention;

FIG. 2a is a front perspective view of a light-emitting head of the therapeutic light source showing panels each carrying an LED matrix;

FIG. 2b is a top view of the light-emitting head showing the direction of illumination;

FIG. 3 is a front view of one of the LED matrices;

FIG. 4 is a circuit diagram showing the series-parallel configuration of each LED matrix;

FIG. 5 shows an emission spectrum of an LED for use in a first embodiment of the invention; and

FIG. 6 shows emission spectra for an LED for use in a second embodiment of the invention.

### DESCRIPTION OF THE EMBODIMENTS

A first embodiment of the invention is directed to healing wounds or damaged tissue using non-coherent polychromatic infrared light. Polychromatic infrared light, for example within the wavelength range 405 nm-904 nm, penetrates the skin and is absorbed by photoreceptors in the cell membrane and mitochondria. The photons create a biochemical response, stimulating singlet oxygen, cellular cytochromes and transient free radical production which results in the formation of proton gradients that facilitates physiological changes resulting in the cessation of pain and a reduction in inflammation and improvement in wound and tissue repair.

Agaiby et. al, ‘Laser modulation of T-lymphocyte proliferation in vitro’, Laser Therapy, 1998, 10, 153-158 has shown the responsiveness of the cellular components of wound healing to photon stimulation. The increase in cellular energy and tissue oxygenation, enhanced microcirculation and synthesis of specialized signaling proteins, such as growth factors, have been shown to be influenced by photons and can lead to the acceleration of wound healing.

Enhanced microcirculation results in an increase in new capillaries as well as new blood vessels, to replace damaged ones. This leads to an increase in the healing process because the vessels can deliver more oxygen and nutrients necessary for healing in addition to an increased removal of waste products. Stimulation of collagen increases the body's capacity to repair damaged tissue and to replace old tissue. Stimulation of adenosine triphosphate (ATP) boosts the transport of energy to all cells. An increase in ATP increases nutrient absorption and waste disposal by cells. An increase in the lymphatic system activity can be brought about by an increase in the lymph vessel diameter and lymph flow rate. Hence, the inventor has realized that modulation of wound healing and/or tissue repair can be achieved by irradiation of the wound or damaged tissue simultaneously by infrared and visible light. Venous and arterial diameters could also be increased in a similar manner.

The absorption of infrared light by photoreceptors within the tissue leads to signal transduction and amplification, and finally results in the photoresponse. Infra red and long-wavelength visible light are absorbed by components of the respiratory chain (i.e. flavine dehydrogenases, cytochromes and cytochrome oxidase), which cause an activation of the respiratory chain and the oxidation of NAS pool which leads to changes in the redox status of both the mitochondria and the cytoplasm. This in turn has an effect on membrane permeability/transport, with changes in the Na′/H′ ratio and increases in Na′/K′-ATPase activity, which in turn has an effect on the Ca++ flux. The Ca++ flux affects the levels of cyclic nucleotides, which modulates DNA and RNA synthesis, which modulates cell proliferation (i.e. biostimulation). Infra-red light initiates the response at the membrane level (probably through photophysical effects on Ca++ channels) at about halfway through the total cascade of molecular events that lead to biostimulation, whereas long-wavelength visible light initiates probably by photoactivating enzymes in the mitochondria, a cascade of molecular events leading to the photoresponse.

The photobiochemical processes involved in wound healing and tissue repair are governed by action spectra. Beauvoit B et. al., ‘Analytical Biochemistry’ 1995, 226, 1670-174 demonstrates that mitochondria provide 50% of the tissue absorption coefficient and 100% of the light scattering at 780 nm due to cytochrome aa3, cytochrome oxidase and other mitochondria chromophores. Karu T, ‘Health Physics’, 1989, 56, 691-704 demonstrates a peak in increased RNA/DNA synthesis by radiation at wavelengths of 633, 680 and 780 nm, increased protein synthesis at 750 nm and increased cell proliferation at 890 nm.

Infrared light can penetrate to a depth of several centimeters, which it makes it more effective for fall-thickness treatment of bones, joints, muscle, etc. Macrophages can inhibit or enhance the activity of many kinds of cells. Light of different wavelengths affects the ability of macrophages to release factors that cause the above effects. Therefore, infra red light therapy has great potential as a modulator of wound repair.

In one embodiment, wounds that are prone to hypertrophy or to keloid formation are treated with wavelengths stimulating the release of inhibiting factors (e.g., prostaglandins) that suppress fibroblast activity. In vitro studies show that 880 nm light had an inhibitory effect on fibroblast proliferation.

In another embodiment, applicable to cases such as varicose ulcers, for example, where the problem is one of delayed repair, wavelengths enhancing the release of stimulating factors (e.g. monokines) are applied to encourage activity and the development of granulation tissue. In vitro studies show that 820 nm and 870 nm light was stimulatory.

A second embodiment of the present invention is directed to a method of treatment of hypopigmentary skin disorders using ultraviolet (UV) or near-UV light.

Low intensity UV irradiation can stimulate melanocytic migration and proliferation, and mitogen release for melanocyte growth, thereby providing a microenvironment for inducing repigmentation in hypopigmentary conditions such as vitiligo; see Tjioe M et al. ‘Acta Derm Venereol’ 2002, 82(5), 369-372. There are very sensitive (near) ultra-violet peaks in the RNA/DNA synthesis/stimulation action spectra at 365, 404 and 434 nm (see the Karu reference cited above), which would result in doses at least an order of magnitude lower than those required at 450, 560 and 633 nm (i.e. 5-50 J/m² compared with 500-5,000 J/m²). There are further peaks around 760 nm which correspond to increased stimulation of ATP.

Apparatus suitable for use in the first and second embodiments is illustrated in FIGS. 1 to 4. A therapeutic light source comprises a base 2, an articulated arm 4 and a light-emitting head 6. The base 2 contains a power supply 3 for supplying electrical power to the light-emitting head 6, and a controller 5 for controlling the supply of power to the head 6. The controller 5 includes a switch and a timer for controlling the switch to determine the interval for which the head is switched on and emits light. The head may be switched on continuously over the interval, or may be pulsed on and off with a periodicity and duty cycle controlled by the controller 5. The interval, periodicity and duty cycle may be programmed into the controller 5 by a user by means of a keypad and display screen (not shown).

The articulated arm 4 is connected to the base 2 by a hinged joint 7a and is articulated along its length by further hinged joints 7b and 7c to give a sufficient degree of freedom in the position and angle of the head 6. The arm 4 carries a power connector from the controller 5 to the head 6.

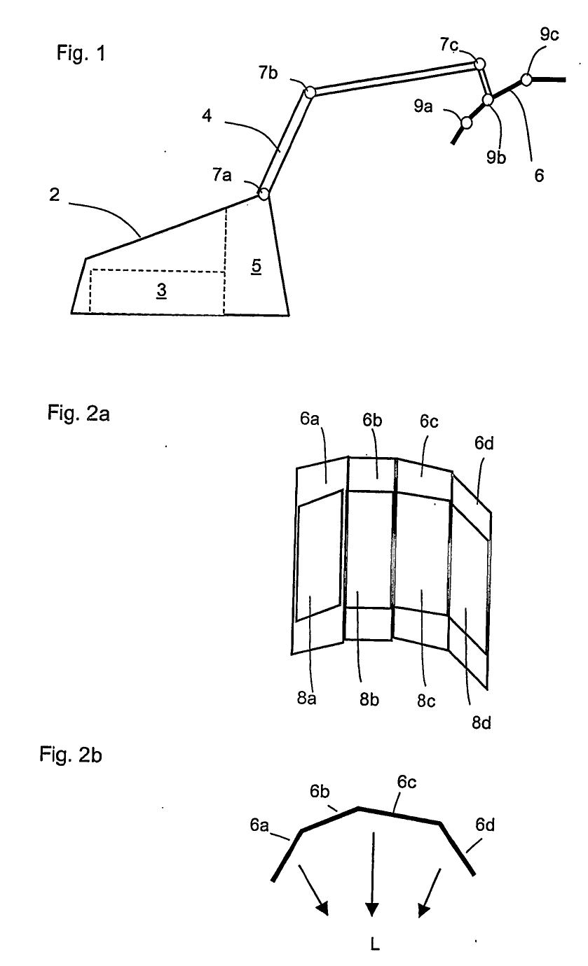

The head 6, as shown more particularly by FIG. 2a, consists of four rectangular panels 6a, 6b, 6c, 6d arranged side by side and joined at their edges by hinges 9a, 9b, 9c. Each panel 6 carries on its front face a corresponding matrix 8a, 8b, 8c, 8d of discrete light-emitting diodes (LED's). As shown in FIG. 2b, the panels 6a-6d can be angled to form a concave surface such that light L emitted by the LED's is concentrated on an area of the patient to be treated.

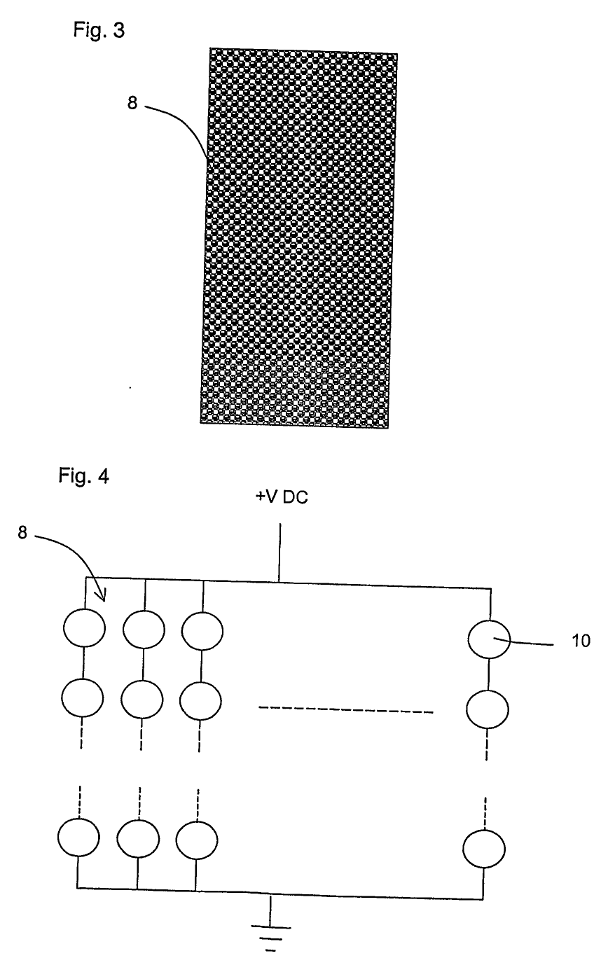

FIG. 3 shows the physical arrangement of LED's in the matrix 8, while FIG. 4 shows the series-parallel electrical connection between the LED's 10. A direct current (DC) voltage +V is applied across the matrix when power is supplied to head 6.

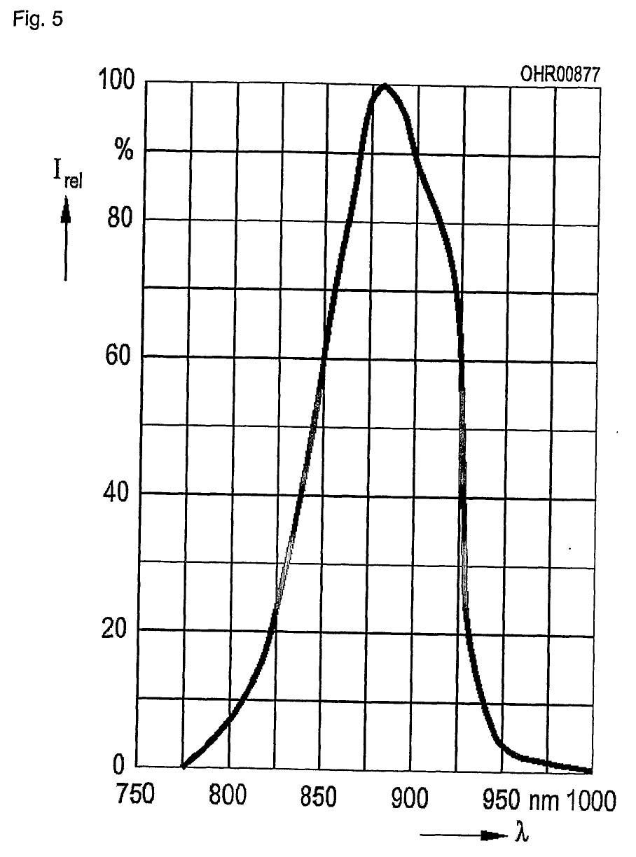

In the first embodiment, the LED's 10 emit in the red and near infrared spectrum, for example substantially in the range 660-950 nm. The LED's may have GaAlAs substrate material. One suitable type of LED is Osram™ part no. SFH 4289, for which the emission spectrum is shown in FIG. 5. The spectrum has a peak at 880 nm. The LED's 10 may be of two or more different types, at least one of which emits in the infrared and one of which emits in the red area of the spectrum. Both types may be switched on together.

Preferably, the combined emission spectra of the LED's 10 have substantial intensity at one, or preferably more than one, of the wavelengths mentioned above with reference to the first embodiment.

The first embodiment may also be applied to a method of cosmetic treatment of the skin.

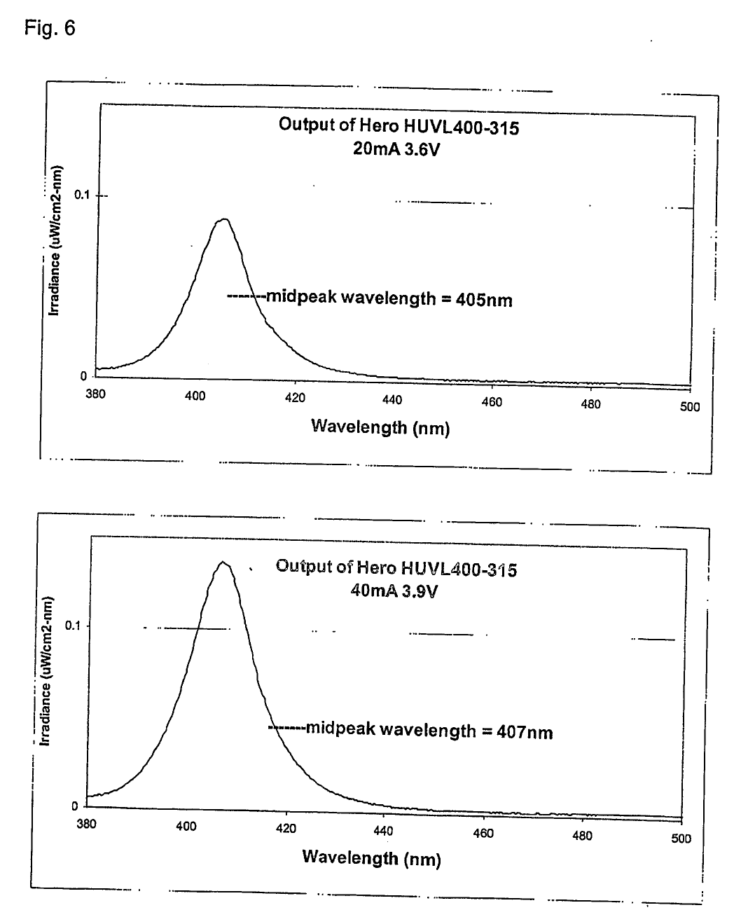

In a second embodiment, the LEDs 10 are ultraviolet (UV) emitting LEDs having emission spectra substantially in the near UV spectrum. The LED's may have InGaN or GaN or InGaN/SiC substrate materials. One suitable type of LED is Hero™ part no. HUVL400-315, having a peak at 400-410 nm, and emission spectra as shown in FIG. 6.

The LED's 10 may be of two or more different types, both of which may be switched on together.

Preferably, the combined emission spectra of the LED's 10 have substantial intensity at one, or preferably more than one, of the wavelengths mentioned above with reference to the second embodiment.

In a method of treatment using the device in the first embodiment, the head 6 is positioned to irradiate an external affected area (e.g. a wound or damaged tissue) of a patient to be treated, which is then exposed to one or more suitable wavelengths at an intensity which is preferably between 1 and 50 mW/cm², but may be between 0.1 and 500 mW/cm². Suitable treatment doses range from 0.5 to 20 J/cm², but could range from 0.1 to 200 J/cm². Treatment times preferably range from 2 to 10 minutes, but may range from 0.5 to 30 minutes. A therapeutic course may consist of up to 30 treatments with intervals between 0.5 to 7 days.

In a method of treatment using the device in the second embodiment, a pseudocatalase (e.g. Vitise™ from SES Derma, Valencia, Spain) may be applied twice daily to boost the low catalase activity often found in hypopigmented patients. Excess pseudocatalase is removed just before light application. The head 6 is positioned to irradiate a hypopigmented area of the skin of the patient to be treated. The patient is then exposed to one or more suitable wavelengths at an intensity which is preferably between 0.1 and 50 mW/cm², but may be between 0.05 and 100 mW/cm². Suitable treatment doses range from 0.01 to 100 J/cm², but may range from 0.05 to 100 J/cm². Treatment times preferably range from 0.5 to 10 minutes, but may range from 0.1 to 30 mins. A therapeutic course consists of up to 100 treatments with intervals ranging from 1 to 7 days.

The above embodiments are provided purely by way of example. Other variants will be apparent from the above description but may nevertheless fall within the scope of the following claims.

1. A therapeutic light source arranged to emit polychromatic light substantially within the wavelength range of 405 to 904 nm.

2. The light source of claim 1, wherein the radiation has substantial intensity at a wavelength of one or more of 633, 680 or 780 nm.

3. The light source of claim 1 or claim 2, wherein the radiation has substantial intensity at a wavelength of 750 nm and/or 890 nm.

4. The light source of claim 1, wherein the radiation has substantial intensity at a wavelength of 880 nm.

5. The light source of claim 1, wherein the radiation has substantial intensity at a wavelength of 820 and/or 870 nm.

6. The light source of claim 1, including an array of discrete light-emitting diodes arranged to emit the polychromatic light.

7. The light source of claim 1, including a plurality of arrays of light-emitting diodes each arranged to emit the polychromatic light, the configuration of the plurality of arrays being adjustable by the user.

8. The light source of claim 7, wherein the plurality of arrays are mounted on an adjustable arm.

9. A method of modulating healing of wounds and/or damaged tissue, comprising irradiating affected tissue with incoherent radiation simultaneously including infrared light and visible light.

10. The method of claim 9, wherein the radiation comprises polychromatic light substantially within the wavelength range of 405 to 904 nm.

11. The method of claim 10, wherein the radiation has substantial intensity at a wavelength of one or more of 633, 680 or 780 nm.

12. The method of claim 10 or claim 11, wherein the radiation has substantial intensity at a wavelength of 750 nm and/or 890 nm.

13. The method of claim 10, wherein the radiation has substantial intensity at a wavelength of 880 nm.

14. The method of claim 13, wherein the wound is of a type prone to hypertrophy or keloid formation.

15. The method of claim 10, wherein the radiation has substantial intensity at a wavelength of 820 and/or 870 nm.

16. The method of claim 15, wherein the damaged tissue arises from delayed repair.

17. The method of claim 16, wherein the tissue is a varicose ulcer.

18. The method of claim 9, wherein the radiation has a total intensity of between 0.1 and 500 mWcm⁻².

19. The method of claim 18, wherein the radiation has a total intensity of between 1 and 50 mWcm⁻².

20. The method of claim 9, wherein the total radiation dose per treatment is between 0.1 and 200 Jcm⁻².

21. The method of claim 20, wherein the total radiation dose per treatment is between 0.5 and 20 Jcm⁻².

22. The method of claim 9, wherein the total time per treatment is between 0.5 and 20 minutes.

23. The method of claim 22, wherein the total time per treatment is between 2 and 10 minutes.

24. The method of claim 1 or claim 9, wherein the radiation is emitted by an array of light-emitting diodes.

25. A method of treatment of a hypopigmentary skin disorder, comprising irradiating a hypopigmented area of a patient's skin with radiation including low intensity ultraviolet radiation.

26. The method of claim 25, wherein the low intensity ultraviolet radiation has substantial intensity at a wavelength of at least one of 365, 404 and 434 nm.

27. The method of claim 25 or claim 26, wherein the radiation has a substantial intensity at a wavelength of approximately 760 mn.

28. The method of claim 25, wherein the total intensity of the radiation is between 0.05 and 100 mW/cm².

29. The method of claim 28, wherein the total intensity of the radiation is between 0.1 and 50 mW/cm².

30. The method of claim 25, wherein the radiation dose per treatment is between 0.01 and 100 J/cm².

31. The method of claim 30, wherein the radiation dose per treatment is between 0.05 and 100 J/cm².

32. The method according to claim 25, including applying a pseudocatalase to the hypopigmented area before the step of irradiation.

33. The method of claim 25, wherein the skin disorder is vitiligo.

34. A method of modulating healing of wounds and/or damaged tissue, comprising irradiating affected tissue with incoherent infrared radiation having substantial spectral intensity at at least one of 820 nm, 870 nm, 880 nm and 890 nm wavelength.
